(19) United States
(12) Patent Application Publication
(10) Pub. No.: US 2008/0269791 A1
(43) Pub. Date: Oct. 30, 2008

(54) KINKED LANCET

(76) Inventors: Joachim Hoenes, Zwingenberg (DE); Frank Deck, Niederkirchen (DE); Hans-Peter Haar, Wiesloch (DE); Uwe Kraemer, Ilvesheim (DE); Volker Zimmer, Laumersheim (DE); Herbert Harttig, Neustadt (DE)

Correspondence Address: BOSE MCKINNEY & EVANS LLP, 2700 FIRST INDIANA PLAZA, 135 NORTH PENNSYLVANIA STREET, INDIANAPOLIS, IN, 46204, US

(21) Appl. No.: 12/126,554

(22) Filed: May 23, 2008

Related U.S. Application Data
(63) Continuation of application No. PCT/EP2006/011341, filed on Nov. 27, 2006.

(30) Foreign Application Priority Data
Nov. 25, 2005 (EP) EP 05 025 739.3

(51) Int. Cl. A61B 17/32

(52) U.S. Cl. 606/181

(57) ABSTRACT

A device for obtaining body fluid is described which has at least one lancet comprising a lancet body and a tip. The lancet has at least one structure with an altered stiffness which can also be referred to as a bending region. This structure preferably has a lower or reduced stiffness than the remaining lancet body such that the lancet can be bent in this region when a force acts thereon. In this process, the orientation of the tip is changed relative to the remaining lancet body. This change in orientation is preferably out of the lancet body plane.

This application is a continuation application of International Application PCT/EP2006/011341, filed Nov. 27, 2006, which claims priority to EP 05 025 739.3, filed Nov. 25, 2005, which are hereby incorporated by reference in their entirety.

### BACKGROUND

The present invention relates to lancing aids for the diagnostic determination of blood parameters. Body fluids are collected and analyzed in many fields of medical diagnostics, and it is therefore desirable to enable routine tests to be carried out outside the laboratory in a rapid and reproducible manner. The testing can be carried out with various body fluids such as, e.g., blood and/or interstitial fluid. These fluids can be examined for various characteristics. The results of this examination are important in order to be able to make reliable diagnoses, to carry out therapeutic measures and for therapeutic monitoring.

The analysis of body fluids starts with collection of the fluid. One method for obtaining body fluid is to generate a very small wound in the skin of the patient with the aid of a needle, lancet or a knife. The body fluid obtained in this manner can then either be collected in small vessels or it can be directly brought into contact with a test element such as a test strip for analysis. In order to avoid injury to the patient when using lancets, needles or blades, the lancing aid is typically constructed with a protector or cover on the lancing tip. Most of these lancing aids require manual insertion of the lancet into the lancing aid. This is a laborious operation when the lancing aid is used frequently. Storage of lancets in a magazine can obviate this problem, but many safety aspects have to be followed. Thus, for example, the safety of the patient during use of the lancing aid must be ensured. Furthermore, the system should not be too complex because otherwise it could not be easily handled by the patient.

A few solutions for this are disclosed in the prior art. U.S. Patent Publication No. 2003/0199902 ensures a sealing of each individual lancet in a magazine in which a complicated and space-filling gear wheel mechanism is used to transport the lancets out of the magazine.

An analytical device is described in EP 1 203 563. This device has a test element on a carrier tape and an additional frame element is mounted on this test element which is movable and comprises a lancet. During use, the frame element can be moved from a parallel position relative to the test element into an orthogonal position so that the lancet can be actuated through an opening in the test element. This is a quite complicated implementation of a combination of test element and lancet because many parts have to be moved mechanically and the system requires much space in its functional form.

EP 1 360 935 describes an arrangement of lancets (referred to as “testers”) used to obtain liquid samples. The lancets are arranged serially on a tape which has a cover for the lancets on its upper side. A complicated mechanical system is used to expose the lancet for use because the entire lancet body must first be moved out of the plane of the tape in order to be able to use the lancet.

These prior art devices present a variety of disadvantages. Many mechanical steps are necessary to move the individual lancing element from the magazine store in which the lancets lie in a serial arrangement, i.e., in the plane of the carrier tape, into an arrangement in which the lancet is arranged perpendicular to the plane of the carrier tape. Due to the complicated mechanics, there is an additional disadvantage that a large amount of space is required for this mechanism. Another disadvantage of many systems of the prior art is the complicated unsealing of the lancet before the lancing operation.

In view of the disadvantages of the prior art, it would be desirable to have a space saving, storable lancing aid that can be used with little mechanical complexity and that enables a simple handling.

### SUMMARY OF THE INVENTION

The invention concerns a device for obtaining body fluid which comprises at least one lancet. The lancet is composed of at least one lancet body and a lancet tip (also referred to as “tip” herein). The lancet has a bending region such that when a force acts on the lancet, it is preferably bent in the region of the tip such that the orientation of the tip is changed relative to the longitudinal axis of the lancet body. The bending of the lancet tip out of the plane of the lancet body is also referred to herein as a first bending movement.

In an exemplary embodiment, the lancet has an elongate extension and one end of which, referred to here as the distal end, is specially shaped for the purpose of insertion into a body, for example in the form of a tip. In this connection, the tip is a point located at the distal end of the lancet into which the side faces of the elongate lancet body converge. The side faces of the lancet which end at the tip can also have sharp edges. Hence, the lancet consists of at least one lancet body which predominantly has almost parallel side faces or edges and a region at the tip or tip region which directly adjoins the lancet body and has side edges which taper towards one another and end at the tip. The region of the tip, or also tip region, can thus be of different sizes depending on the length of the side edges which taper towards one another.

In order to bend the lancet tip the lancet has a bending region. This bending region provides at least one site in the lancet which is suitable for bending at least the lancet tip using the smallest possible amount of force. This bending region can extend over parts of the lancet tip region as well as over parts of the lancet body. The bending region has at least one structure with a changed or reduced stiffness relative to the remainder of the lancet, which allows the lancet to be easily deformed at this site, i.e., the lancet is bent along the bending region. The bending region preferably begins in the tip region and extends over a part of the lancet body, the width of which represents an extension of the tip region. The lancet body has two substantially parallel edges. The other part of the lancet body which adjoins the bending region and merges into the proximal or rear end of the lancet, can have a geometry that is different from the tip region such as, for example, a widening or thickening of the lancet body. This broader part of the lancet body can additionally be made harder in order to have a higher resistance to deformation. This can, for example, be accomplished by the selection of other materials or by suitable choice of the amount or thickness of the materials used.

If the lancet is mounted on a carrier, then at least the rear part of the lancet body, which preferably has a structure that is more rigid towards deformation, can be connected with the carrier in order to make a stable connection with the carrier.

The rear part of the lancet body can also be used for coupling to a drive unit. For this purpose, the lancet body can have various coupling structures such as, for example, grooves, notches or protuberances. The drive preferably takes place transversely to the alignment of the lancet body such that the bent lancet tip can be linearly inserted into a body part. The orientation of the bent lancet tip, which is different from that of the longitudinal axis of the lancet body, has the advantage that more geometric arrangements can be implemented than would be possible with a purely axial drive direction for the lancet body and lancet tip. Furthermore, due to the exemplary bending of the lancet tip, which, due to its intended use (of being inserted into a body part as painlessly as possible), the lancet in the bending region should be designed to be as thin and fine as possible, so that only a small amount of force is necessary or a small morphological change of the lancet has to be carried out in order to enable an easy bending. This ensures a simple bending of the lancet without increasing the lability of the lancet when it is stored on a carrier or carrier tape. The lancet body, which is used for the coupling to the drive unit, can be made to be stable independently of the design of the bendable tip region in order to withstand the strains when the drive unit is coupled to the lancet and produces forces on the lancet during the lancing movement. Bending of the lancet outside of the bending region could destabilize the lancet structure.

At least the lancet tip can be provided with a sterile protection (protector) which is preferably opened or pierced when the lancet is bent.

In one embodiment, the lancet is located on a carrier. The carrier can, for example, be used for the simple storage of a plurality of lancets. In addition, the carrier can also have the function of protecting the lancet against external influences (such as, for example, knocks or other contacts) when the lancet is at least partially surrounded by the carrier. This is particularly useful when the carrier is a carrier tape. In an exemplary embodiment, the lancet body and the lancet tip are attached in an unbent state to the carrier and preferably in a lying position in which the lancet is arranged horizontally or flat against the carrier. At least the tip region rests completely on the carrier.

A circular structure to which or on which the at least one lancet is fastened can be used alternatively as a carrier. The carrier preferably has a disk-shaped design. However, other carrier structures which, for example, have square, spherical or tape like, oval or elliptical shapes are possible.

The lancet and the carrier can be formed in one piece. This is preferred when the entire structure is produced from metal such as steel. However, other materials such as ceramics or polymer structures would also allow the lancet and carrier to be formed in one piece.

In an alternative embodiment, the lancet comprises a structure which is suitable for taking up body fluid. This can be a capillary structure, but alternative structures such as hole structures, gap or groove structures are suitable for taking up liquid. In this case a structure formed by stamping in the bend region, preferably in the tip region, can be designed to take up liquid. This embodiment is referred to in the following as a microsampler because the sample is taken up by the lancet and not directly by a test element. The structure for taking up body fluid can be preferably located in the tip region. In an alternative embodiment it can also extend beyond the tip region and extend over parts of the lancet body. The structure for taking up body fluid can be formed in one piece or divided into several regions. In an exemplary embodiment, this structure for taking up liquid begins in the tip region and extends into the lancet body to almost the same extent as in the tip region. In this case, the structure for taking up body fluid can open out into the bend region or protrude into the lancet body beyond the bend region. The body fluid collected in the microsampler can be subsequently transferred to a test element and detected by a detection system (e.g., optically or electrochemically) and evaluated by an evaluation system.

In addition, at least one test element can be arranged in, on or next to the carrier or a further carrier. The test element is used to take up the body fluid obtained and to subsequently detect an analyte in the body fluid. The test element can contain reagents for reaction with the analyte. The test element can be attached to a separate carrier or to the carrier of the lancet. In order to prevent contamination of the tip with substances from the test element, the test element is preferably not directly connected to the lancet but is rather arranged on the carrier separately from the lancet. The arrangement of the at least one test element on a separate carrier reduces the risk of contamination. In an exemplary embodiment, the test element and lancet are arranged relative to one another such that after the lancet tip has been bent, they can be brought into contact by a second movement of the lancet or of the test element. This is particularly useful when the lancet is designed as a microsampler. This can be carried out by a movement of the lancet relative to the carrier. One method of bringing the lancet into contact with the test element is to further bend the lancet in the first bending direction such that the lancet tip is bent by more than 90° with reference to the lancet body. In this embodiment the test element is preferably located on a part of the lancet body. An alternative movement of the lancet for contacting the lancet with a test element is a deflection movement of the lancet or of the carrier in the opposite direction to that of the first bending movement. In this movement, the lancet tip can be bent back into the plane of the lancet body. As an alternative to contacting the test element with the lancet, the body fluid can also be directly transferred from the body part of the user onto the test element.

Another alternative for contacting the test element with the lancet is to move the test element itself. For this purpose the test element can be located on a second carrier, in which case the carrier of the lancet or of the test element are arranged such that they can at least partly be moved relative to one another.

In one embodiment, the carrier is designed as a carrier tape on which a plurality of lancets are positioned. In this embodiment, a device for obtaining body fluids is described which has an essentially planar carrier tape with a longitudinal orientation and a transverse orientation on which at least one lancet comprising a lancet body and a tip is arranged, the lancet is being arranged substantially horizontally on the carrier tape. As already described, the device is characterized in that the lancet comprises a structure with a changed (typically reduced) stiffness (compared to the remaining lancet material) which, as already mentioned, is referred to as the bending region.

The stiffness should be understood as a measure of the resistance of the material to elastic deformation. This structure should preferably have a lower stiffness than the remaining lancet body so that the lancet can be bent preferably in this region under the action of force. In this process, the orientation of the tip changes relative to the remaining lancet body. This change in orientation is preferably away from the carrier tape plane or lancet body plane. In this process, at least a part of the lancet body remains in its original plane or in the carrier tape plane and can be attached thereto. The force which is required to change the orientation of the lancet tip is also referred to as the threshold force. This threshold force should be of a sufficient magnitude that it changes the orientation of the lancet tip but in so doing, is such that no unintentional deformations occur on the lancet, on the carrier or on the carrier tape.

Force can be transferred to the lancet by a bending element, e.g., a push rod which is pressed onto the lancet. In one embodiment having more than one indentation, the force can be transferred onto the lancet by guiding the carrier or the carrier tape with the lancet over the push rod. In this case, a force of sufficient magnitude (threshold force) acts on the lancet in order to move the lancet tip out of the lancet body plane or out of the carrier tape plane. In the case of lancets on a carrier or carrier tape, at least a remainder of the lancet body remains on or attached to the carrier tape.

In another embodiment, the bending element is in two parts. In this case the lancet tip is conveyed by the push rod out of the carrier tape plane or the lancet body plane due to the fact that the lancet body itself or together with the carrier tape is prevented from moving in the direction of the push rod movement by the second part of the bending element. This second part of the bending element can be a stop which is located on the side of the carrier, carrier tape or lancet that is opposite to the push rod. The push rod as well as the stop can additionally be controlled by a control element such that the position of the bend can be varied. In this manner the lancet can be bent at various positions and tips of different lengths are formed for puncturing the skin. The transfer of force from the push rod to the lancet is particularly simple when the tip region of the lancet is not permanently connected to the carrier or carrier tape. In this connection a flat lancet is particularly suitable for the arrangement on a carrier or carrier tape.

The bending region of the lancet, which can also be outside the tip region, has at least one structure with a modified stiffness. This at least one structure with a modified stiffness is referred to as an indentation, as already mentioned. The indentation can be worked into or onto the lancet by for example stamping or hammering or other metal processing measures. The stiffness can thus be adjusted by varying the geometry of a component or by varying the amount of material in the component. One embodiment comprises more than one indentation in the bending region of the lancet. A particular embodiment of this indentation is a triple indentation in the bending region of the lancet which extends over at least a part of the longitudinal extension of the lancet. In this case one indentation extends from the distal end of the lancet in an axial direction towards the proximal end of the lancet. The length of the indentation is variable. This indentation can be introduced into the flat lancet from two sides. This difference in the direction of the indentation has the effect that the lancet tip bends away in the opposite direction to the lancet body.

The first part of the first indentation is located in the tip region. This first part of the indentation can be limited to the tip region but can also extend beyond this region. A second indentation adjoins the proximal end of the first part of the first indentation in the direction of the side edge of the lancet. A third indentation also adjoins the proximal end of the first part of the first indentation and extends towards the opposite side edge of the first indentation of the lancet. The second and third indentations are impressed from the same side as the first part of the first indentation. These indentations provide multiple axes along which the lancet can bend. The orientation of these indentations enables the areas bordering on the at least one indentation to be bent or reoriented by means of a low threshold force on the lancet. As a result, the bent areas are lifted from the lancet body plane or carrier tape plane at an angle of preferably up to 100°. As a result, the lancet tip is moved out of the plane of the lancet body or of the tape.

The material of the lancet is typically metal such as steel. The lancet can, however, also consist of other materials which enable the lancet to be bent when a force acts on it and have sufficient stiffness to be able to penetrate the skin during use without changing shape. Furthermore, the material can be such that the distal end of the lancet can be worked into a sharp tip because otherwise too much pain would be generated during the puncture. The manufacture of lancets is in general sufficiently known in the prior art such as for example in DE 19 604 156 or EP 0 565 970.

The carrier tape is preferably produced from a plastic foil. It can, however, also be another more flexible material as described, for example, in U.S. 2005/0245845. In an integrated system, at least one test element can be additionally arranged on the carrier tape. The lancet and test element can be provided in an alternating arrangement. The lancet can be attached to the tape diagonally, in a longitudinal orientation as well as in a transverse orientation. One possible embodiment is to arrange the lancet and test element in direct vicinity of one another. This enables direct transfer of liquid onto the test element after the lancing operation without having to move the tape further.

Various methods are described herein for actuating the lancet. The proximal end of the lancet can be attached to the carrier or carrier tape in such a manner that part of the lancet can be moved relative to or with the carrier or carrier tape whereas the proximal end remains connected to the carrier or carrier tape at one or more points. Another attachment of the lancet is to secure the lancet body on the carrier or carrier tape, whereby the tip region detaches from the carrier or carrier tape. The lancet can be moved in a controlled manner by moving the carrier or carrier tape or by gripping the lancet with a gripper element as a result of which the lancet is moved with the carrier or carrier tape from the plane of the carrier or of the carrier tape. This movement can be executed by means of a drive element which transfers force onto the lancet perpendicular to the carrier or carrier tape plane. The force is transferred by a drive element which can, for example, be a push rod or a gripper element which grips and moves the body of the lancet. In this connection the puncture depth of the blood withdrawal device can be freely selected in one embodiment. In order to adjust the puncture depth, the movement of the lancet is defined by a variable stop element against which the lancet impacts during the lancing operation. In this manner the length of the lancet tip which emerges from the housing opening and thus the lancing depth is varied as a function of the position of the stop element. The stop element can be integrated into the housing. Furthermore, the lancet can itself be used as a stop element where the lancing depth is defined by the length of the bent tip. Since in the bent state the lancet is bent at an angle to the lancet body which differs from 0°, the lancet body can represent a barrier for the further penetration of the lancet into the skin. Thus, it is possible to use lancets having several indentations in the bending region in order to select the puncture depth by the choice of the particular indentation that is used for the bending.

The lancet can be driven by ballistic or sliding block-guided mechanisms which are well-known in the art and are described, for example, in DE 19 604 156, EP 0 565 970, U.S. Pat. No. 5,318,584 or U.S. Pat. No. 4,924,879. One embodiment for the lancet drive is the free movement of the lancet after force has been transferred by a drive element such as a push rod. In this embodiment, an impulse is transferred from a drive element onto the lancet and the lancet moves without further guidance by the drive element towards the housing opening. The movement of the lancet can be guided by additional elements on the housing.

In order to use the system hygienically, the lancet is protected by a sterile protection at least in the tip region. The lancet can be covered by a protective foil over the entire lancet body. The foil can also extend over a part of the carrier tape or carrier and is connected thereto. This sterile protection can consist of a polymer layer which is applied after connecting the lancet to the carrier tape or carrier. The sterile protection is destroyed or pierced by the lancet tip when the threshold force is applied to the lancet tip to thus expose a part of the lancet, at least the tip region of the lancet. Alternatively, the sterile protection can be removed before using the lancet. In this case the entire sterile protection is preferably removed.

The invention also concerns a system for obtaining body fluid. This system has a housing in which a carrier or a substantially planar carrier tape is mounted, and at least one lancet which is arranged horizontally on the carrier or carrier tape. The housing has at least one opening through which the lancet tip can pass when it is actuated. The substantially planar carrier tape can be wound onto two spools. However, it is also possible to use other storage methods to store the used and unused lancets as already described, e.g., in the form of a carrier. If two spools are used to store the lancets, the unused lancets are on one spool and the used lancets are on the other spool. The lancets consist of a material which is soft enough to be wound onto the carrier tape without being bent in this process. On the other hand, the material of the lancets is sufficiently stable that the lancet is not deformed when it is actuated and when it penetrates the skin. Alternatively, the lancets are arranged transversally on the carrier tape to avoid bending of the lancet. Another method of avoiding bending of the unused lancets is the selection of the diameter of the spool on which the lancets are stored to be sufficiently large that the lancets are hardly bent when the spool is rolled up.

The lancet has a lancet tip which is located on the distal end of the lancet. A bending element is present in the system which acts on the lancet such that the orientation of the lancet tip can be changed relative to the remaining lancet body. In one embodiment, the bending element can control the position at which the force acts on the lancet body when force is exerted on the lancet before actuation. For this purpose, the bending element can be controlled by a control element. A push rod can be used for the transfer of force.

Another embodiment of the bending element is a push rod over which the lancet, the carrier or the carrier tape with the lancet is passed such that force which acts on the lancet in this process is sufficient to bend the lancet tip. In order to achieve good force transfer to the lancet, the carrier tape can be tensioned. Other embodiments of a one-piece bending element for the carrier tape include a wheel with a radius that is as small as possible (see FIG. 4a) or a guide over an edge (see FIG. 3) over which the carrier tape is passed. The mode of operation of these alternatives is described in more detail in the detailed description.

Conventional lancets (typically flat lancets) and other lancets in which the threshold force of the bending element is sufficient to move the tip of the lancet out of the plane of the carrier tape or of the lancet body plane can be used in the system or the device. The lancet is moved by a drive element towards a housing opening after or during the bending operation in order to subsequently carry out the lancing operation. In this process at least a part of the lancet emerges from the housing opening and punctures the skin of the patient. A drop of blood forms at the puncture site which is used for analysis. If a test element is located on the carrier or carrier tape, the carrier or the carrier tape is transported if necessary by such a distance that the test element is located below the housing opening. The drop of blood can be applied to the test element without the patient having to initiate further steps. Alternatively, the test element can also be located on a second carrier as already described. The blood reacts with one or more reagents which are located on the test element such as those that are known from EP 0 885 591, EP 0 535 480, and EP 0477322. The test element is analyzed by means of a detector.

The blood can be examined for various components as is known in the art. For example, the analysis can be for blood components such as hematocrit, glucose, cholesterol, coagulation, iron and others. Various methods can be used for the analysis. For example, electrochemical detection reactions can be used, but also optical (e.g. reflection, absorption, fluorescence, Raman-spectroscopy) or magnetic detection reactions. The liquid is typically brought into contact with a test system and a reaction takes place between a test element and the liquid. Thus, detection by means of an optical test element is based on a color reaction between the liquid and detection reagent. Examples of these reactions are described in U.S. Pat. Nos. 3,802,842; 4,061,468 and 4,490,465.

When the instrument is in use the system carries out various steps. The lancet is brought into a position in which it can be brought into the bent state by the action of a threshold force on the lancet body. In this process the sterile protection is preferably pierced by the lancet. If necessary, the lancet is transported to the opening of the housing. There it is actuated with the aid of a drive element, and part of the lancet thus emerges from the housing opening. During the actuation operation, at least a part of the lancet penetrates the skin of the patient and is afterwards retracted into the device. If a microsampler is used, blood can be collected on the lancet in this process. If a transport tape is used, this is transported further and wound onto a spool. In this case, the lancet preferably again lies flat on the carrier tape. This process of re-storage is described in U.S. Patent Application No. 2005/0245845.

In an integrated system in which test elements are also attached to the carrier or carrier tape in an alternating arrangement with the lancets, the test element is transported after the lancing operation to the housing opening in order to take up the drop of blood for analysis. The test element can be transported up to the detector and measured there. If a microsampler is used, the collected blood is transferred to a neighboring test element. As already mentioned, the test element can be present on the same carrier or on a second carrier. In this connection, the two carriers can be arranged such that they can be moved relative to one another.

### BRIEF DESCRIPTION OF THE DRAWINGS

The above-mentioned aspects of the present invention and the manner of obtaining them will become more apparent and the invention itself will be better understood by reference to the following description of the embodiments of the invention, taken in conjunction with the accompanying drawings, wherein:

FIG. 1a is a schematic representation of a lancet with an indentation positioned in an unused state;

FIG. 1b is a schematic representation of a lancet of FIG. 1a in a bent state;

FIG. 1c is a schematic representation of a flat lancet having several indentations positioned in an unused state;

FIG. 1d is a schematic representation of the lancet of FIG. 1c in a bent state;

FIG. 1e is a perspective view of the lancet shown in FIG. 1d;

FIG. 2a is a schematic representation of lancets positioned transversely on a carrier tape;

FIG. 2b is a schematic representation of lancets positioned in a longitudinal arrangement on a carrier tape;

FIG. 3 is a schematic representation of a lancet and tape and its guidance in a longitudinal section;

FIG. 4a is schematic representation of the actuation of a lancet by guiding the carrier tape over a roller;

FIG. 4b is a schematic representation of the actuation of the lancet in which the tape is moved horizontally out of its plane;

FIG. 5a is a schematic representation of a carrier tape having an alternating arrangement of test fields and lancets arranged longitudinally;

FIG. 5b is a schematic representation of a carrier tape having an alternating arrangement of test fields and lancets, the lancets arranged transversely;

FIG. 6 is a schematic representation of an integrated device with a housing and other components;

FIG. 7 is a schematic representation of a multilancet wheel shown from a top view; and

FIGS. 8a-d are perspective views of the lancets from FIG. 7 in an unbent state and in three different stages of bending.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

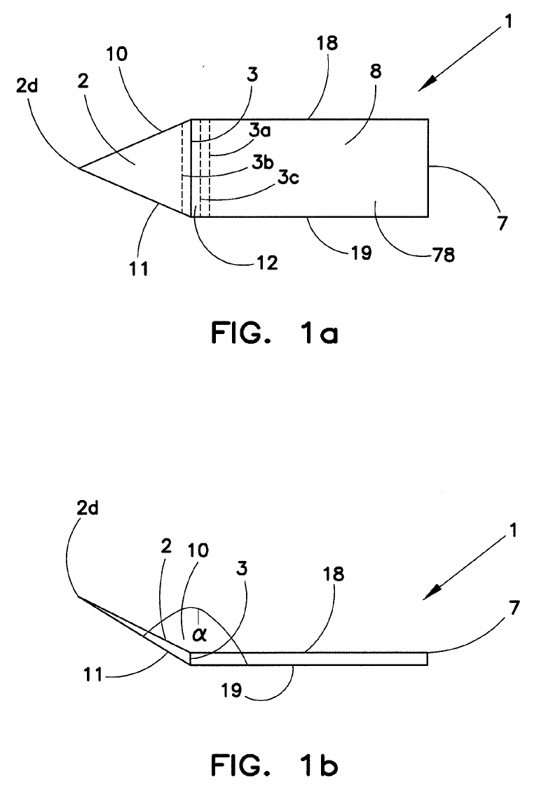

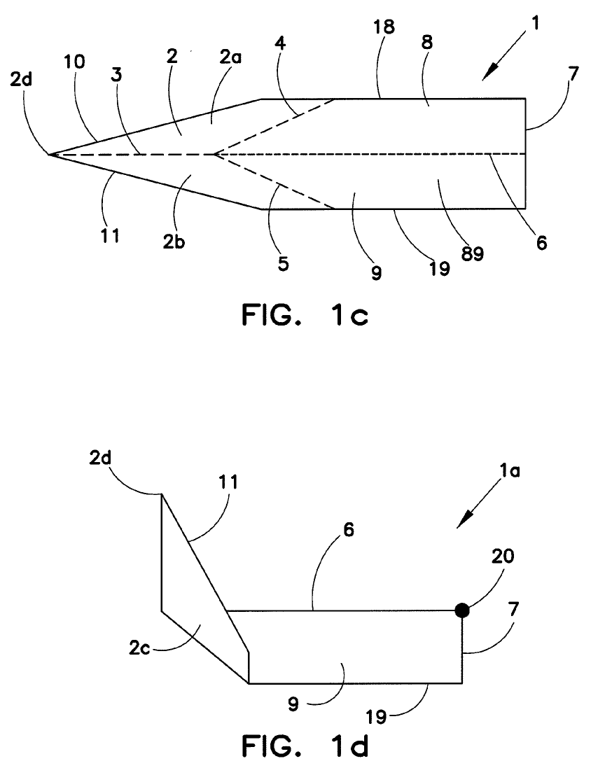

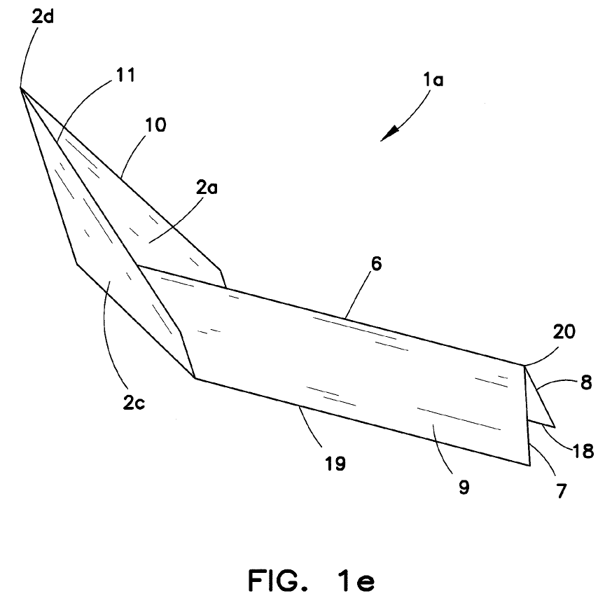

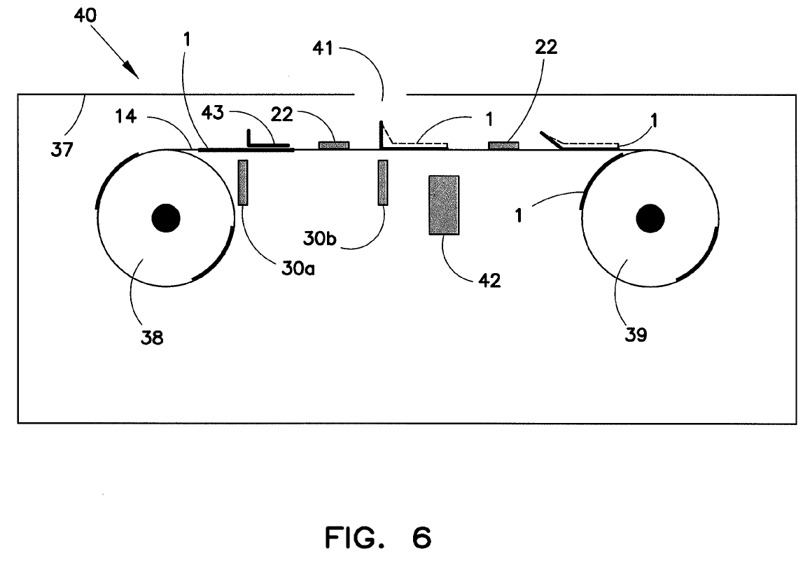

FIG. 1a shows a possible embodiment of the lancet 1. The lancet 1 has a distal end 2 and a proximal end 7. The lancet 1 has a region 8 which adjoins the proximal end 7 and merges into the tip region 2. The lancet 1 has a structure 3 having a modified stiffness which is referred to in the following as an indentation. The indentation 3 is located in the bend region which can be within or outside of the tip region 2. This indentation 3 can be located at three different sites 3a, 3b, 3c between the distal and proximal end of the lancet. The bending region 12 is defined by the region of the indentations 3, 3a, 3b, 3c and can vary depending on the number and position of the indentations. At least one indentation 3, 3a, 3b, 3c is within the tip region 2. The bending region can additionally extend over a part of the lancet body 78. In this connection, the bending region adjoins the tip region 2. The tip region 2 ends at the lancet tip 2d. When the lancet is bent, a bending element which is controlled by a control element not shown here, see FIG. 6 has the effect that the lancet is bent at one of its indentations 3, or 3a, 3b, 3c. Depending on the position and selection of the bend the lancet 1 can penetrate the skin to different depths during the lancing operation.

A lancet 1 with an indentation 3 is shown in a bent state in FIG. 1b. The lancet tip 2d as well as the tip region 2 are angled at an angle a relative to the region 8. The angle a can be between 180° and about 80°. A preferred range is between 150° and 110°.

A lancet 1 which represents one embodiment of the invention is shown in FIG. 1c. This lancet 1 preferably has at least one structure with an altered stiffness. This altered stiffness helps to facilitate and direct the change in the orientation of the lancet tip relative to the remaining lancet body. The stiffness is preferably lowered or reduced in this structure so that when a force acts on the lancet body, the tip region 2 with the lancet tip 2d bends at the site of reduced stiffness. Such a structure with an altered stiffness can be made by various methods. For example, during the manufacturing process of the lancet 1, less material can be incorporated at this site. Another possibility would be a punching or hammer process for introducing the structure or a stamping process. In addition, metal processing methods known in the art which result in a structure having an altered stiffness can be used. In one exemplary structure, the first indentation is impressed into the material from the distal end 2d of the lancet 1 at least over part of the tip region 2 towards the proximal end 7 of the lancet 1. This indentation 3 can extend to the proximal end 7 of the lancet. A second indentation 4 can be introduced into the material laterally to this indentation. This indentation can begin inside or outside the tip region 2 and extends from the middle 6 of the lancet 1 towards the side edge 18. The angle between this indentation 4 and the midline 6 of the lancet 1 α is between 0° and 90°. This angle a is preferably between 30° and 70°. A third indentation 5 extends on the side opposite to the indentation 4. This indentation 5 also extends from the middle 6 of the lancet 1 towards the side edge 19. The angle between the middle line 6 and the indentation 5 is also between 0° and 90° and a preferred range is between 30° and 70°. The angles α and β must not be identical. The transition of the side edges 10 and 11 into the side edges 18 and 19 of the lancet forms the border of the tip region. The two side edges 10 and 11 meet at the tip 2d. The bending region can be within and also outside of the tip region 2 of the lancet, and in a preferred embodiment, extends over the entire length of the lancet 1. In order to be able to introduce the indentations into a lancet 1, a flat lancet is preferably used which is composed of a thin metal sheet.

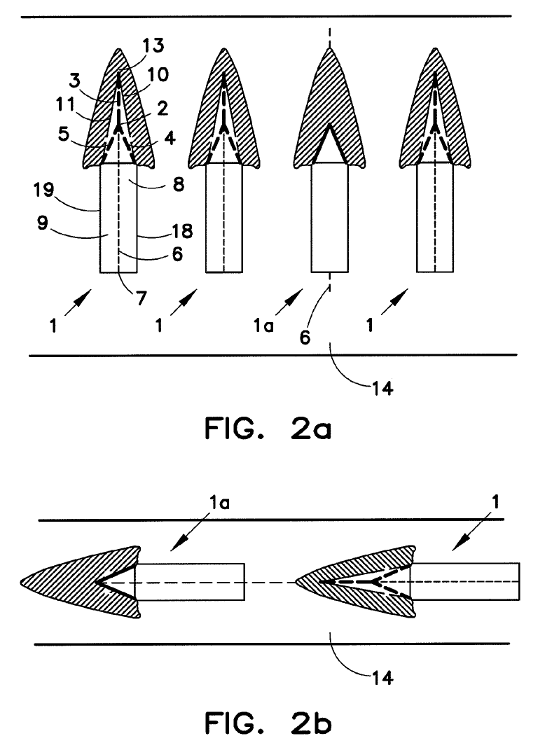

Bending lines are formed by the indentations 3, 4, 5 in the sheet metal. These bending lines result in an at least partial bending of the metal sheet to one side and partially to the other side of the lancet plane 89 of FIG. lc. That is, the lancet bends along multiple axes such as indentations 3, 4, and 5 when a force is applied. The lancet plane 89 is formed by the surfaces 8 and 9 of the unbent lancet 1. In FIG. 1c the lancet plane 89 is in the plane of the paper. A side view of the bent lancet is shown in FIG. 1d so that the lancet plane 89 is rotated by up to 90° from the plane of the paper. A perspective view of a bent lancet is shown in FIG. 1e. A directed bending of the lancet 1 can also be achieved by perforation or scoring or etching along the lines 3, 4, 5. Due to the special arrangement of the bending lines 3, 4, 5, the tip 2d is bent upwardly by up to 90° relative to the lancet surface 89 when a force acts on the lancet and simultaneously the side faces 2a and 2b of the tip region 2 are bent and the faces 8 and 9 of the remaining lancet body are bent downwardly in the opposite direction, as best seen in FIG. 2e. In this embodiment, the lancet body has a high stability despite the structure with a reduced stiffness. The lancet 1 is sufficiently stable to carry out a lancing operation into the skin of a patient to obtain blood. In the unbent state, before or after a puncture, the lancet can be preferably rolled onto a tape as described in U.S. Patent Application 2005/0245845. The puncture can, for example, be carried out by rotating the lancet around the lancet end 20. The indentations 3, 4 enclose an area 2a, whereas the indentations 3, 5 enclose an area 2b. In the embodiment example shown in FIG. 1c, indentations 4, 5 end outside the tip region 2 which is delimited by the side edges 10, 11. These lines extend beyond the tip region to the edges 18, 19 up to the proximal end 7 of the lancet 1. In one embodiment, further indentations parallel to the indentations 4 and 5 can be worked into the bending region (not shown in the FIGS.) in addition to the indentations 4 and 5. With the aid of these alternative indentations it is possible to bend the tip at different positions and thus the tip can have different lengths, depending upon which of the alternative indentations are selected for the bending. This enables different puncture depths into the skin.

The lancet is shown in a bent state in FIGS. 1d and 1e. During the bending process, the faces 2a, 2b are bent upwards out of the lancet plane 89 whereas the faces 8, 9 are moved downwards from the plane 89, as shown in FIG. 1e. In this process, the midline 6 preferably remains in the lancet plane 89. The lancet tip 2d can be bent by up to 100° relative to the face of the lancet plane 89. As a result, the underside of the tip region 2 becomes visible as shown in FIGS. 1d and 1e as the face 2c. This forms the rear side of the face 2b in FIG. 1c. The faces 8 and 2a are not visible in the side-view of FIG. 1d, but are visible in the perspective view of FIG. 1e. In FIG. 1d, the face 8 lies behind the face 9 whereas the face 2a is hidden behind face 2c. When the lancet 1 is rotated clockwise around the pivot point 20, the lancet 1 executes an upward movement and when rotated in the reverse direction it moves downward. An alternative drive of the lancet 1 would be to grip the lancet shaft at the proximal end 7 of the lancet 1 with a gripper or pliers. In this case the lancet 1 would not be rotated around a point 20 but rather moved as a whole. In this embodiment the carrier tape 14 should have sufficient flexibility in order not to limit the movement of the lancet.

FIG. 2a shows the lancets 1 after their manufacture. In this embodiment, the lancets 1 are fashioned out of a thin tape of sheet metal which in this case represents the carrier tape 14 by punching or etching. The lancets 1 are arranged transversely on the carrier tape 14. They have indentations 3, 4 and 5 in the tip region 2 and the indentations 4 and 5 end at the proximal end of the edges 10 and 11. A hollow space 13 extends around the tip region which is produced by punching out or etching. This hollow space 13 in the carrier tape 14 around the tip region 2 enables the tip 2d to be bent out of the lancet plane as shown for one lancet 1a. The tip 2d of this lancet 1a is bent upward and the lancet midline 6 represents the bending line.

In FIG. 2b the lancets 1 are arranged in a longitudinal orientation on the carrier tape 14. FIG. 2a shows an unbent lancet 1 as well as a bent lancet 1a. In the case of the bent lancet 1a the lancet tip 2d is bent out of the carrier tape plane 14.

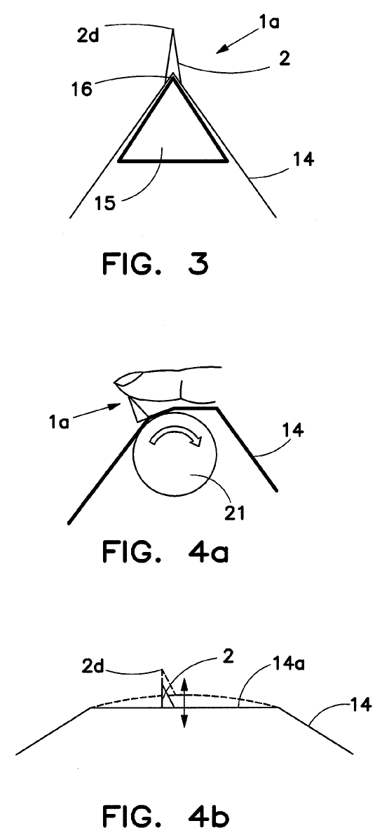

FIG. 3 is a schematic representation of the arrangement of lancet 1 relative to the carrier tape 14 and to a guide 15 of the carrier tape 14. In this case the guide 15 is shown as an equilateral triangle where the carrier tape 14 is guided over an edge 16. It can be seen here that as soon as the lancet 1 reaches the deflection edge 16, the lancet tip 2d is bent out of the carrier tape plane. This method of bending lancets can be used for the lancets that are described in FIG. 1c.

FIG. 4a shows a further method of guiding the carrier tape 14 in such a manner that the lancet tip 2d is automatically bent out of the carrier tape plane 14. In this case the carrier tape 14 is guided over a roller 21 which, depending on the arrangement of the carrier tape 14 on the roller 21, can either be rotated to the right or to the left. In this embodiment, the lancet 1 extends in a substantially longitudinal orientation on the tape 14 and the proximal end 7 of the lancet 1 moves in front of the lancet tip 2d. The roller 21 can, for example, consist of a profiled wheel which prevents the carrier tape 14 from slipping on the roller 21. In this case, the lancing operation occurs by a rapid forward and backward rotation of the wheel 21. In this case, the bending of the lancet 1 can be facilitated by an additional bending element (e.g., a bulge, not shown here) on the wheel 21. This bulge exerts a force on the middle 6 of the lancet body in addition to the force caused by the rotation of the wheel 21. This force bends the side faces 8 and 9 of the lancet body downwardly and thus bends the lancet tip 2d upwardly.

A bent lancet 1a is shown in FIG. 4b whose tip 2d projects from the carrier tape plane 14a. In order to carry out the lancing operation, the tape 14 is moved in the direction of the lancet tip 2d by a bolt not shown here on the side opposite to the lancet 1a. Another method of moving the lancet 1a is with the aid of a gripper (not shown) which grips the shaft of the lancet 1a and executes the lancing operation by means of an up and down movement. One embodiment for this lancing operation is to tension the tape 14 before the lancing operation. The elasticity of the carrier tape should preferably be chosen such that it can be deflected by the lancing depth (about 2-3 mm). The deflection of the carrier tape can be altered by varying the force acting on the carrier tape and thus the lancing depth (or the distance by which the lancet emerges) can be varied.

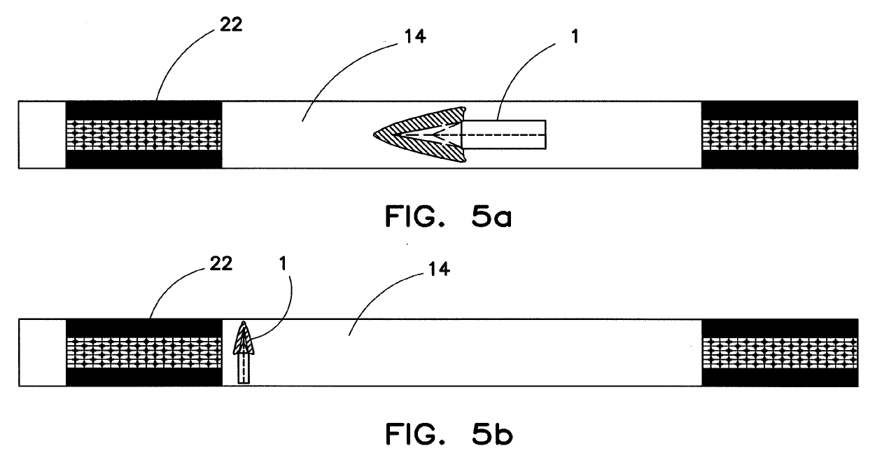

A carrier tape 14 is shown in FIG. 5a on which test fields 22 and a lancet 1 are arranged alternately. The lancet 1 is aligned longitudinally relative to the carrier tape. The distance between the test field 22 and the lancet 1 on the carrier tape 14 can vary. Thus, it is possible that the lancet 1 is placed sufficiently close to the next test field 22 that after the puncture, the liquid can be immediately taken up by the test field 22 without moving the carrier tape 14. Another embodiment with alternating test fields 22 and lancets 1 is shown in FIG. 5b. In this case the lancet 1 is arranged transversely on the carrier tape. Also in this case the lancet 1 can be placed at a variable distance to the test field 22.

An integrated system is shown in FIG. 6. The system consists of a device 40 which preferably has a housing 37 with an opening 41 as well as a carrier tape 14 on which the lancets 1 are attached. The carrier tape 14 is wound onto two spools 38 and 39. The unused portion of lancets attached to the carrier tape are on spool 38 and the used portions are wound onto spool 39. The carrier tape 14 is stretched between the spools 38, 39. The spools 38, 39 are moved by a drive such as those known in the art. Only one of the two spools 38, 39 need be driven. An example of such a drive is described in U.S. patent application 2005/0245845. The lancets 1 are in an unbent state on the carrier tape 14 when the carrier tape 14 is wound onto the spools. A first push rod 30a is located between the two spools 38, 39 and is used to transfer force onto the lancet 1 for the bending process. This push rod 30a is located below the carrier tape 14. In order that the force is not used only to deflect the carrier tape 14, a bending element 43 which impedes the vertical movement of the carrier tape 14 is located above the carrier tape 14 opposite the push rod 30a. The bending element 43 can have different shapes depending on the arrangement of the at least one indentation on the lancet. In the case of a lancet having only one indentation no particular shape of the bending element is necessary because in this embodiment the bending element only has the function of preventing further movement of the lancet with the carrier tape. In one embodiment of the bending element 43 in which a lancet having more than one indentation should be bent as shown in FIG. 1c, the bending element 43 has a shape which, although preventing movement of the faces 8 and 9 to be bent, does not prevent movement of the bending region with the at least one indentation 6. An example of this arrangement is a bending element 43 with two wings which extend above the faces to be bent but sufficient space is left between the wings so that the lancet can move further into this space and be bent.

The bending element 43 can comprise a control element not shown here which can change the position of the bending element 43 in such a manner that the lancet is bent in different places. This is particularly preferred in embodiments which utilize only one indentation 3 or 3a, 3b, 3c for bending the lancet tip.

A second push rod 30b which is located below the housing opening 41 is used to drive the lancet during the actuation. The carrier tape 14 is located between the housing opening 41 and push rod 30b. In order to trigger the lancing process, the carrier tape 14 is transported until an unused lancet 1 is situated between the housing opening 41 and push rod 30b. When the lancing operation is triggered, the push rod 30b is moved onto the lancet 1 with so much force that at least the lancet tip 2 is moved out of the housing opening 41. After the puncture is completed the blood is collected on a test field 22. Here a reaction takes place between the blood and the reagents on the test field which can be analyzed with the aid of a detector 42. The lancet 1 is re-stored together with the carrier tape 14. As a result of the winding process on the spool 39, the lancet is again integrated in a flat manner into the carrier tape 14.

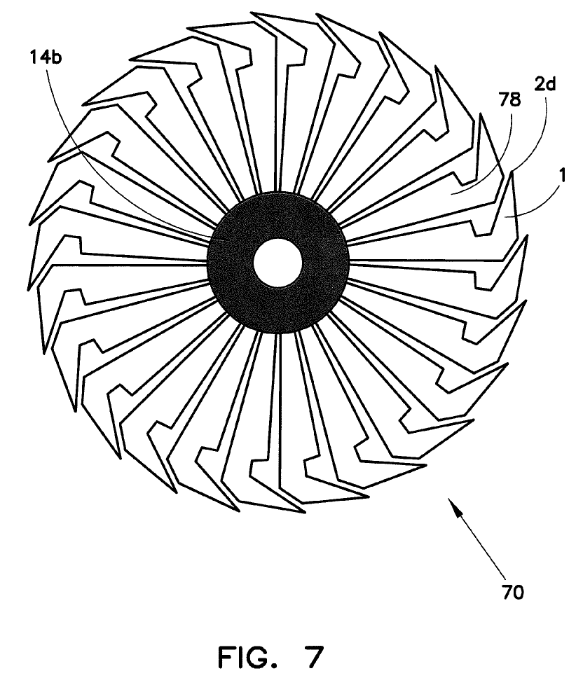

FIG. 7 shows a further method of storing a plurality of lancets 1 in the form of a multilancet wheel 70 in which the lancets 1 are arranged in one plane. In this case the lancet tips 2d are fashioned into a sabre shape such that the lancet tip 2d has to be bent sideways in order to use the lancets 1. For this purpose, an indentation is introduced into the tip region 2, which is preferably approximately parallel to the lancet body 78 (not shown). A test element can be additionally mounted on the lancet wheel 70 (not shown). In addition, the lancet 1 can be designed as a microsampler. For this purpose, it then preferably has a structure for taking up body fluid, preferably in the tip region 2. Also not shown is a further indentation which can be located in the transition region between the tip region 2 and lancet body 78 which enables a further bending of the lancet tip 2 after the lancing operation. This enables body fluid to be transferred onto a test element which can also be situated on the carrier or lancet body.

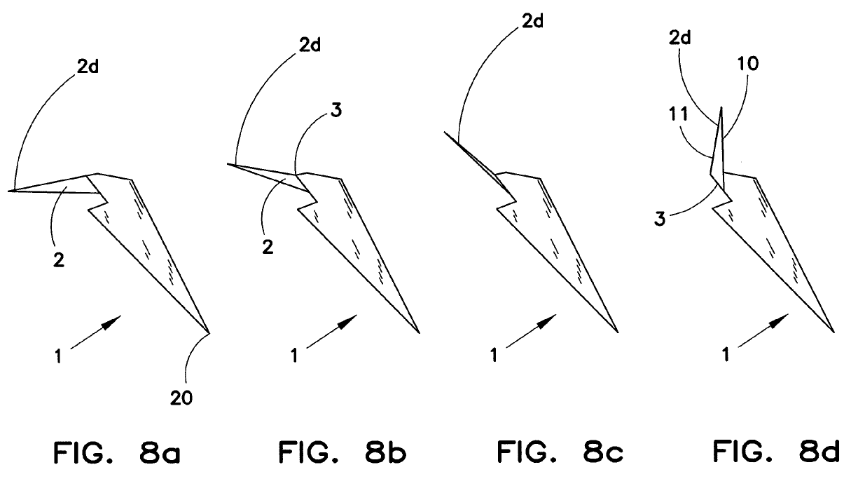

The sideways bending is shown in FIGS. 8a-d. The lancet 1 is shown in an unbent state in FIG. 8a. FIGS. 8b and c show the lancet 1 during the bending process during which the lancet tip 2d is slowly bent out of the lancet body plane 89 and is ready for lancing at a certain angle as shown in FIG. 8d. During the lancing, the lancet 1 is moved in a circle around the point 20. Due to this circular movement it is advantageous to adapt the shape of the bent tip region 2 to the circular movement, which means that the edges 10 and 11 are shaped such that they ensure a puncture or incision in the skin with the least possible pain. Due to the position of the tip region 2 in relation to the region 8 of the lancet 1 it can be seen that when the lancet 1 is rotated around the point 20, the edges 10 and 11 penetrate the skin perpendicular to the cutting movement whereas when a lancet 1 is used as described in FIG. 1d, the edges 10 and 11 impact the skin perpendicularly to the lancing movement at an angle which differs from 0 degrees.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A device for obtaining body fluid, comprising:
a substantially planar carrier tape;
at least one lancet arranged substantially horizontally on the carrier tape, the lancet comprising a lancet body and a tip; and
the lancet comprising a bending region, wherein a force applied to the lancet bends the lancet in the bending region and reorients the tip relative to a longitudinal axis of the lancet body.

2. The device of claim 1, wherein the at least one lancet is substantially flat.

3. The device of claim 1, wherein the reorientation of the tip comprises the tip projecting from the substantially planar carrier tape.

4. The device of claim 1, wherein the bending region comprises a region of reduced stiffness.

5. The device of claim 4, wherein the region of reduced stiffness extends to the tip.

6. The device of claim 4, wherein, when the force is applied to the lancet, areas adjoining the bending region are reoriented.

7. The device of claim 1, wherein the at least one lancet is aligned substantially parallel to a longitudinal direction of the carrier tape.

8. The device of claim 1, wherein the at least one lancet is aligned substantially transverse to a longitudinal direction of the carrier tape.

9. The device of claim 1, further comprising at least one test element arranged on the carrier tape.

10. The device of claim 1, wherein a proximal end of the lancet is secured to the carrier tape.

11. The device of claim 1, wherein the tip comprises a sterile protector.

12. The device of claim 11, wherein the sterile protector is separated from the lancet tip during the reorientation.

13. The device of claim 1, wherein the bending region comprises a plurality of bending regions angled relative to one another.

14. A system for obtaining body fluid, comprising:
a substantially planar carrier tape;
at least one lancet arranged substantially horizontally on the carrier tape, the lancet comprising a lancet body and a tip; and
a bending element operable to exert a force on the lancet, the force changing the orientation of the tip relative to the lancet body.

15. The system of claim 14, wherein the bending element comprises a control element that bends the lancet at different locations in a bending region of the lancet.

16. A device for obtaining body fluid, comprising:
a lancet having a tip and a lancet body; and
the lancet comprising a bending region, wherein a force applied to the lancet bends the lancet in the bending region and reorients the tip relative to a longitudinal axis of the lancet body.

17. The device of claim 16, further comprising a carrier on which the lancet is arranged.

18. The device of claim 17, wherein the carrier is substantially disk shaped.

19. The device of claim 17, wherein the carrier and the lancet are formed in one piece.

20. The device of claim 17, wherein, prior to use, the lancet comprises an unbent configuration in which the lancet is arranged substantially horizontally on the carrier.

21. The device of claim 20, wherein the tip extends from the carrier when the tip is reoriented.

22. The device of claim 21, wherein the tip returns to a substantially horizontal arrangement on the carrier after use of the lancet.

23. The device of claim 17, further comprising a test element arranged on the carrier.

24. The device of claim 23, wherein the lancet and the test element are arranged side by side.

25. The device of claim 24, wherein the lancet and the test element make contact after the reorientation of the tip as a result of a second movement of the lancet or the test element.

26. The device of claim 16, further comprising a sterile protector that covers or surrounds the lancet tip before the tip is reoriented.

27. The device of claim 26, wherein the sterile protector is destroyed or pierced when the lancet tip is reoriented.

28. The device of claim 16, wherein the bending region is located in the tip and in a part of the lancet body which is configured to be coupled to a drive unit.

29. The device of claim 28, wherein the lancet comprises a drive direction that is substantially parallel to the reoriented lancet tip and substantially orthogonal to a plane defined by the lancet body.

30. The device of claim 16, wherein the bending region comprises a plurality of regions with reduced stiffness.

31. The device of claim 30, wherein the length of the bent tip can be selected.

32. The device of claim 16, wherein the lancet comprises a curved saber shape.

33. The device of claim 16, wherein the lancet tip is configured to draw body fluid.

34. The device of claim 33, wherein the tip comprises a capillary.

35. A method of withdrawing body fluid from a body part using a bendable lancet having a lancet body and a tip, the lancet being supplied in a substantially horizontal arrangement on a substantially planar carrier, the method comprising:
applying a force to the lancet, wherein the lancet bends in a bending region thereof and the tip is reoriented to project away from the carrier; and
puncturing the body part with the tip.

36. The method of claim 35, wherein the lancet body remains attached to the carrier during the applying of the force to the lancet.

37. The method of claim 35, further comprising returning the tip to the substantially horizontal arrangement on the carrier after the puncture.

38. The method of claim 37, further comprising rolling the used lancet and a portion of the carrier to which the lancet is connected onto a spool.

39. The method of claim 35, wherein, during the applying of the force to the lancet, a portion of the lancet body bends downwardly as the tip is reoriented upwardly.

40. The method of claim 35, wherein the lancet bends along multiple axes during the applying of the force to the lancet.

41. The method of claim 35, further comprising transferring a body fluid sample obtained from the puncture from the tip to a test element arranged on the carrier.

42. The method of claim 35, wherein, during the applying of the force to the lancet, the tip becomes separated from a sterile protector.

43. The method of claim 42, wherein, during the applying of the force to the lancet, the tip pierces the sterile protector.

44. The method of claim 35, further comprising drawing a body fluid sample into the tip by capillary action.

45. The method of claim 35, further comprising exerting the force on the lancet with a bending element.

46. The method of claim 45, further comprising bending the lancet in a plurality of different locations.

47. The method of claim 35, wherein the puncture comprises driving the lancet in a puncture direction that is substantially parallel to the reoriented lancet tip and substantially orthogonal to a plane defined by the carrier.
